(19) United States
(12) Patent Application Publication
(10) Pub. No.: US 2010/0145344 A1
(43) Pub. Date: Jun. 10, 2010

(54) METHOD AND SYSTEM FOR COMPUTER ASSISTED SURGERY FOR BICOMPARTMENTAL KNEE REPLACEMENT

(75) Inventors: Jason Sean Jordan, Hernando, MS (US); Christopher Patrick Carson, Seymour, CT (US)

Correspondence Address: DIANA HOUSTON;SMITH & NEPHEW, INC., 1450 BROOKS ROAD, MEMPHIS, TN, 38116, US

(73) Assignee: Smith & Nephew, Inc., Memphis, TN (US)

(21) Appl. No.: 12/527,397

(22) PCT Filed: Feb. 14, 2008
(86) PCT No.: PCT/US08/54003

§ 371 Date: Feb. 16, 2010

Related U.S. Application Data
(60) Provisional application No. 60/889,876, filed on Feb. 14, 2007.

(51) Int. Cl. A61B 17/58

(52) U.S. Cl. 606/88

(57) ABSTRACT

A method of resecting distal and anterior portions of a distal portion of a femur for a bicompartmental prosthesis is provided. The method includes generating a geometric representation of the distal portion of the femur. A virtual anterior resection plane is calculated at a predetermined depth and is oriented at a predetermined angle relative to the femur. The method identifies a distal-most point of a lateral portion of the virtual anterior resection plane and an AP line. A varus/valgus angle and an anterior-posterior distance are calculated. Anterior and distal resection guides are navigated according to the parameters calculated from the method.

### REFERENCE TO RELATED APPLICATIONS

This application claims the benefit of U.S. provisional application 60/889,876 filed Feb. 14, 2007.

### BACKGROUND

1. Field of Related Art

The present invention relates to computer assisted surgery. More particularly, the invention relates to computer assisted surgery for partial knee prosthesis.

2. Background of Related Art

Systems for computer assisted surgery for total knee replacement and unicondylar systems are known. A total knee replacement, also known as a tricompartmental knee replacement, replaces both the medial and lateral condyles on the femur and the intracondylar region of the femur where the patella contacts the femur, known as the trochlear groove. The patella may also be replaced in this total knee replacement system. During the surgical procedure for a total knee replacement, knee ligaments are generally cut prior to implantation so that the knee may be accessible for the surgeon. A unicondylar knee replacement system replaces either one of the condyles (a unicondylar prosthesis) or the trochlear groove (a patellofemoral prosthesis.)

Bicompartmental knee replacement surgery, replacing either of the condyles and the trochlear groove, allows one of the anatomic condyles to remain intact through the surgery. In addition, the bicompartmental replacement may be a ligament saving alternative to a total knee replacement. The surgical methods for a bicompartmental knee replacement use jigs and guides attached and positioned to the femur and tibia through the intramedullary canals of the femur and tibia. Guides which invade the intramedullary canal may be more invasive than surgical procedures using computer assisted surgery to position cutting jigs relative to the bone and may increase the risk of fat embolisms.

### SUMMARY

In one embodiment, a method of resecting distal and anterior portions of a distal portion of a femur is provided. The method includes generating a geometric representation of the distal portion of the femur. Another step creates a virtual anterior resection plane within the geometric representation of the distal portion of the femur at a predetermined depth on the anterior portion of the distal femur. The virtual anterior resection plane is oriented at a predetermined angle relative to the femur in internal/external rotation. The method selects the distal-most point of a lateral portion of the virtual anterior resection plane. Another step identifies an AP line on the geometric representation of the distal portion of the femur. The method calculates a varus/valgus angle between a plane perpendicular to the mechanical axis of the femur and a plane passing through the distal-most point of the lateral portion and the AP line. A step measures an anterior-posterior distance between a posterior portion of a condyle of the femur and the intersection point. Another step navigates an anterior resection guide perpendicular to the AP line at a depth determined by the anterior-posterior distance between the posterior portion of the condyle of the femur and the intersection point. The method includes navigating a distal resection guide oriented according to the varus/valgus angle.

In an alternative embodiment, the condyle of the femur may be the medial condyle.

In an alternative embodiment of the method, the geometric representation may be calculated from a point cloud.

Alternatively, the geometric representation may be calculated from an MRI.

Yet another alternative embodiment includes a method wherein the depth of the distal resection is further adjusted according to flexion-extension balancing.

An alternative embodiment provides a system for resecting distal and anterior portions of a distal portion of a femur. The system includes a geometric representation of the distal portion of the femur. A virtual anterior resection plane within the geometric representation of the distal portion of the femur at a predetermined depth on the anterior portion of the distal femur is provided. The virtual anterior resection plane may be oriented at a predetermined angle relative to the femur in internal/external rotation. The virtual anterior resection plane includes a distal-most point of a lateral portion of the virtual anterior resection plane and an AP line on the geometric representation of the distal portion of the femur. The geometric representation has an anterior-posterior distance between a posterior portion of a condyle of the femur and the intersection point. Computer code may be configured to calculate a varus/valgus angle between a plane perpendicular to the mechanical axis of the femur and a plane passing through the distal-most point of the lateral portion and the AP line. An anterior resection guide may be navigated perpendicular to the AP line at a depth determined by the anterior-posterior distance between the posterior portion of the condyle of the femur and the intersection point. A distal resection guide may be navigated according to the varus/valgus angle.

An alternative system provides the condyle of the femur may be the medial condyle.

In another embodiment, the geometric representation is calculated from a point cloud.

In another embodiment, the geometric representation may be calculated from an MRI.

Alternatively, the distal resection is further adjusted according to flexion-extension balancing.

In yet another embodiment, fiducials attached to the anterior and distal resection guides and fiducials attached to the femur are provided.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is an example of a bicompartmental knee prosthesis;

FIG. 2 is an example of an anterior resection guide for a femur;

FIG. 3 is an example of a distal resection guide for a femur; and

FIG. 4 is a flowchart of steps for cutting a femur according to an aspect of the invention.

### DETAILED DESCRIPTION OF THE EMBODIMENTS

The following description of the preferred embodiment(s) is merely exemplary in nature and is in no way intended to limit the invention, its application, or uses.

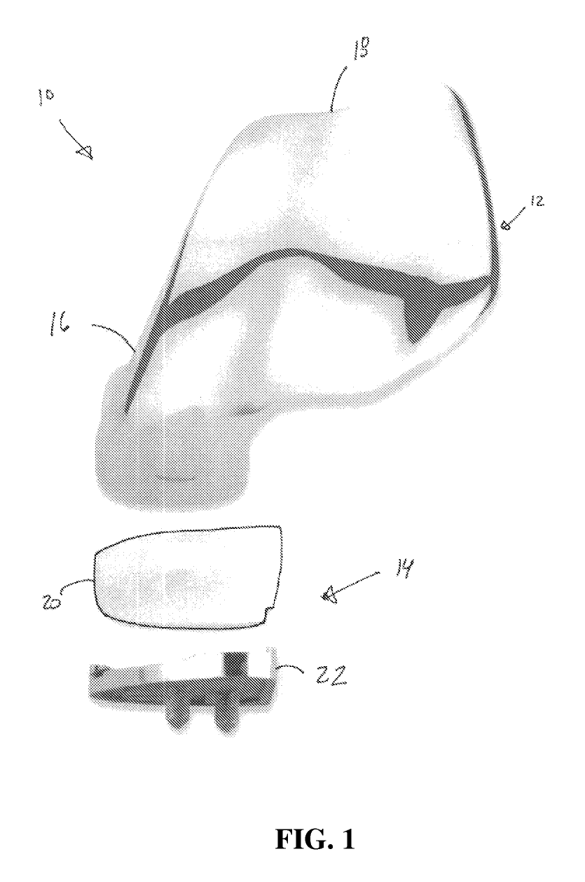

Turning now to the drawings figures, FIG. 1 is an example of a bicompartmental knee prosthesis 10. The prosthesis 10 includes a femoral component 12 and a tibial component 14. The femoral component 12 includes a condylar portion 16 and a trochlear groove portion 18. The tibial component 14 includes an articulating surface 20 and a tibial tray 22.

The femoral component 12 is configured to overlay a femoral condyle and the trochlear groove. While the prosthesis 10 of FIG. 1 is shown as a medial condyle bicompartmental prosthesis, a lateral condyle bicompartmental prosthesis would similarly overlay one femoral condyle and the trochlear groove. The shape of a medial condyle bicompartmental prosthesis may be different than the shape of a lateral condyle bicompartmental prosthesis. In either embodiment, the femoral prosthesis 10 is configured to generally approximate the natural shape of the femur.

The tibial component 14 includes the articulating surface 20 and the tibial tray 22. The tibial tray 22 is configured to attach to the tibia and support the articulating surface 20. The articulating surface 20 is generally shaped to contour to the condylar portion 16 of the femoral component 12. The articulating surface 20 may be made, for example, from a polyethylene material which may promote minimal frictional interference between the articulating surface 20 and the femoral component 12. The articulating surface 20 allows for rotation of the femoral component 12 relative to the tibial component 14 while providing a surface to transmit force components from the femoral component 12 to the tibial component 14.

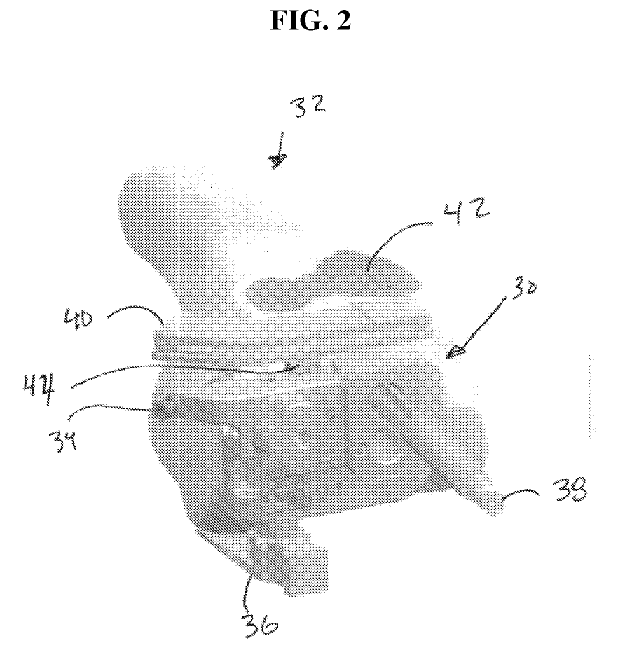

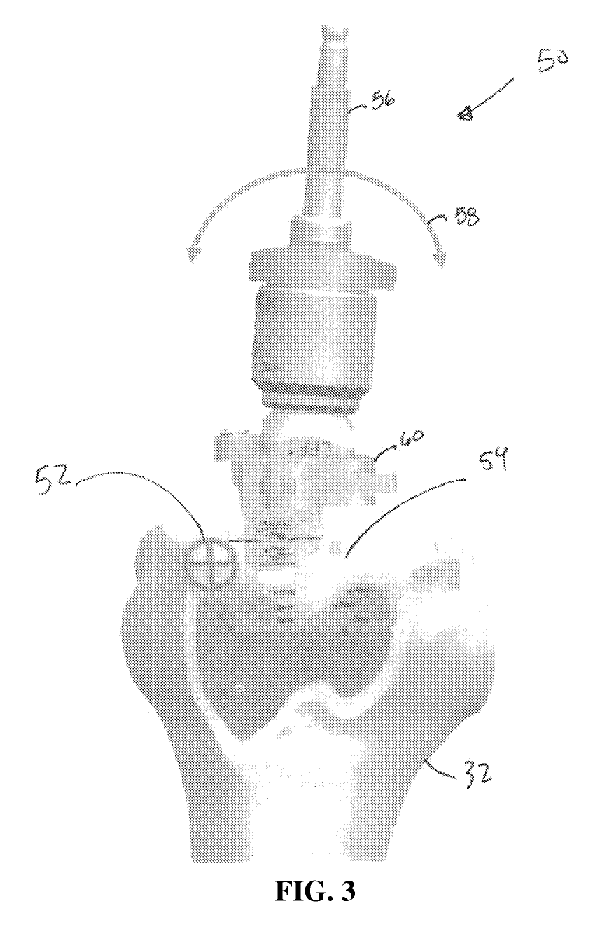

In order for the femoral component 12 and the tibial component 14 to be placed on the natural femur and tibia, bone must be removed from the femur and tibia. When the bone is removed, the components 12 and 14 may be recessed flush to the natural bone surrounding the components 12 and 14. The geometries of the bone are very complex and vary from individual to individual. When cutting bone away from the femur and tibia, variables such as cut depth, cut angles (in all directions) and cut length must be considered. Resection guides, as shown in FIGS. 2 and 3, set these cutting variables when implemented in a computer aided surgical system.

Turning now to FIG. 2, FIG. 2 is an example of an anterior resection guide 30 for a femur 32. Retaining components, such as a pin 34 and a paddle 36 position and place the anterior resection guide 30 relative to the femur 32. A distal pin 38 may also position and place the resection guide relative to the femur 32. The positioning and placing components 34-38 are configured to locate the anterior resection guide 30 in a position where a knife guide 40 is located to take a recessed portion 42 of femur 32 from the anterior surface of the femur 32. The positioning and placing components 34-38 set the angular directions of the anterior cut. A depth gauge 44, attached to the knife guide 40 sets the depth of the anterior cut.

Turning now to FIG. 3, FIG. 3 is an example of a distal resection guide 50 for the femur 32. A transition point 52 defined by the computer assisted surgical system is the point from which the distal cut originates. A paddle 54 against the anterior cut orients the distal resection guide 50 relative to the anterior cut. A valgus collet 56 orients an angular shift defined by the direction 58 about an alignment guide 60 of the distal resection guide 50. The distal resection guide 50 is positioned to cut the distal portion of the femur 32 at an angle from the transition point 52 extending medially as the cut moves from the anterior surface to the posterior surface. The valgus collet 56 orients the valgus alignment of the cut. Together, the interaction of the anterior resection and the distal resection determine the transition zone between the prosthesis and the natural bone.

Together, the resection guides 30 and 50 position and orient the anterior and distal resections. These resections form the basic cuts for placing the prosthesis. The guides 30 and 50 are sized such that the transition from the surface of the implant to the native bone of the femur 32 is generally continuous. In order to make the prosthetic generally continuous, the resection guides must be placed according to the geometry of the femur 32. The geometry may be determined by a point cloud representation of the surface generated through CT scans, MRIs or other scanning techniques. The geometry also may be represented by specific reference points referenced from the CT scans, MRIs or other scanning techniques. The calculated geometry, when isolated within the computer assisted surgical system and transferred to the physical geometry of the femur, for example, through fiducials attached to the physical geometry of the femur and attached to the guides 30 and 50, allow for proper placement of the resection guides which results in proper resection of the femur 32. The fiducials may be registered within the computer aided surgical system in order to properly orient and locate the resection guides 30 and 50 relative to the femur. After the resected anterior and distal portions of bone are removed, then a femoral cutting block may be used to make the additional cuts which make the bone conform to the interior of the prosthetic 10.

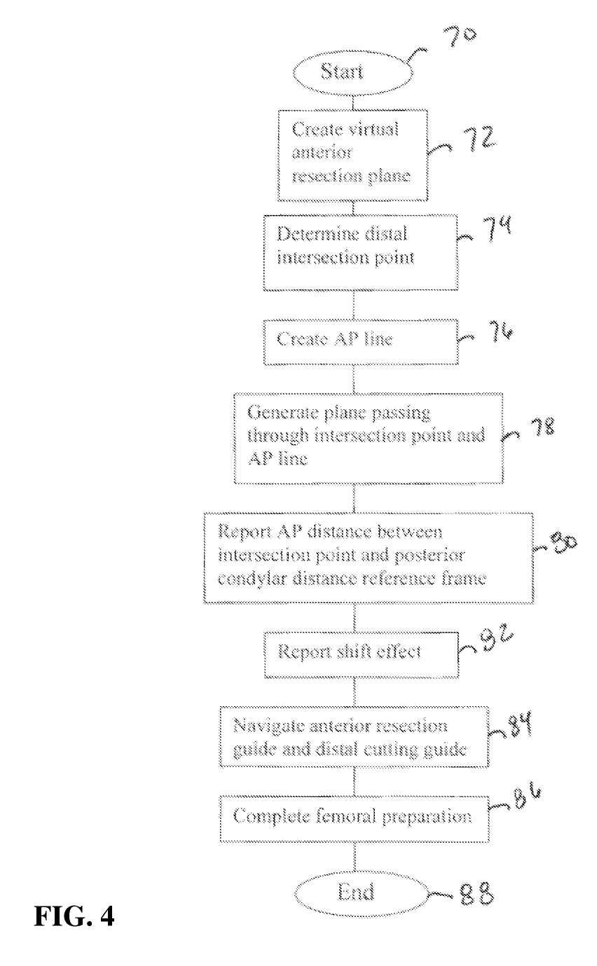

Turning now to FIG. 4, FIG. 4 is a flowchart of steps for cutting a femur according to an aspect of the invention. The method starts in step 70. A virtual anterior resection plane is created in step 72. Step 74 determines the distal intersection point. The AP line is created in step 76. A plane passing through the intersection point and the AP line is generated in step 78. Step 80 reports the distance between the intersection point and the posterior reference frame. Step 82 reports shift effects. From the calculations of steps 72-82, the anterior resection and distal resections are navigated in step 84. In step 86, the femoral preparation is completed. The method ends in step 88.

In step 72, the virtual anterior resection plane is created tangent to the anterior cortex. The plane is used to determine the surface points where the anterior resection would intersect the surface of the femur. The intersection point in step 74 is calculated from the virtual anterior resection plane of step 72 and an articular point cloud calculated from the femur geometry to determine the most distal intersection point of the articular point cloud and the virtual anterior resection plane. The AP line is also calculated from the point cloud in step 76. The AP line is calculated tangent to the trochlear groove at the most proximal point.

The intersection point and the AP line are together used to define a plane in step 78. The angle between this plane and a mechanical axis is reported. This plane may be used to determine valgus angles and may be used to determine an angle relative to a distal point determined by a point cloud reference frame of the distal condyles at the most distal point of the distal point cloud. This distal reference frame is used to determine the anterior-posterior distance to the intersection point. In step 82, the shift effect anteriorly or posteriorly on valgus angle and distal resection are reported. All of the calculations are used to position and place anterior and distal resection guides for resections. The guides are navigated in step 84 so that a smooth transition zone between the implant and the lateral distal cartilage of the femur. The finishing cuts of the femoral cutting block on the anterior and distal resections are made in step 86 and the femoral preparation is completed. The method ends in step 88.

The method and devices described above allow for a femoral preparation to be completed without the need of using an IM rod, which may increase the risk of fat embolism. The accuracy of placement of the anterior and distal resections may be increased as the resections are calculated prior to making either resection. This also may allow for proper placement of the device in the transition zone between bone and implant and proper calculation of the transition point.

While the system and method has been described relative to a femoral component, similarly, the method and system may be used to calculate the tibial resection, and may calculate the tibial resections relative to the femoral preparation. In addition, additional imaging methods such as ultrasound may be used in performing the geometric calculations for the resections.

For example the femoral preparation may include generating a geometric representation of the distal portion of the femur. Another step creates a virtual anterior resection plane within the geometric representation of the distal portion of the femur at a predetermined depth on the anterior portion of the distal femur. The virtual anterior resection plane is oriented at a predetermined angle relative to the femur in internal/external rotation. The method selects the distal-most point of a lateral portion of the virtual anterior resection plane. Another step identifies an AP line on the geometric representation of the distal portion of the femur. The method calculates a varus/valgus angle between a plane perpendicular to the mechanical axis of the femur and a plane passing through the distal-most point of the lateral portion and the AP line. A step measures an anterior-posterior distance between a posterior portion of a condyle of the femur and the intersection point. Another step navigates an anterior resection guide perpendicular to the AP line at a depth determined by the anterior-posterior distance between the posterior portion of the condyle of the femur and the intersection point. The method includes navigating a distal resection guide oriented according to the varus/valgus angle.

In a specific embodiment, the condyle of the femur may be the medial condyle, and the geometric representation may be calculated from a point cloud or an MRI. The depth of the distal resection may be further adjusted according to flexion-extension balancing.

An alternative embodiment provides a system for resecting distal and anterior portions of a distal portion of a femur. The system includes a geometric representation of the distal portion of the femur. A virtual anterior resection plane within the geometric representation of the distal portion of the femur at a predetermined depth on the anterior portion of the distal femur is provided. The virtual anterior resection plane may be oriented at a predetermined angle relative to the femur in internal/external rotation. The virtual anterior resection plane includes a distal-most point of a lateral portion of the virtual anterior resection plane and an AP line on the geometric representation of the distal portion of the femur. The geometric representation has an anterior-posterior distance between a posterior portion of a condyle of the femur and the intersection point. Computer code may be configured to calculate a varus/valgus angle between a plane perpendicular to the mechanical axis of the femur and a plane passing through the distal-most point of the lateral portion and the AP line. An anterior resection guide may be navigated perpendicular to the AP line at a depth determined by the anterior-posterior distance between the posterior portion of the condyle of the femur and the intersection point. A distal resection guide may be navigated according to the varus/valgus angle.

A specific embodiment may provide the condyle of the femur is the medial condyle. The geometric representation may be calculated from a point cloud or an MRI. The distal resection may be further adjusted according to flexion-extension balancing. Fiducials may be attached to the anterior and distal resection guides and fiducials may be attached to the femur.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

The following is claimed:

1. A method of resecting distal and anterior portions of a distal portion of a femur, comprising the steps of:
generating a geometric representation of the distal portion of the femur;
creating a virtual anterior resection plane within the geometric representation of the distal portion of the femur at a predetermined depth on the anterior portion of the distal femur, the virtual anterior resection plane being oriented at a predetermined angle relative to the femur in internal/external rotation;
selecting an intersection point as the distal-most point of a lateral portion on the geometric representation of the virtual anterior resection plane;
identifying an AP line on the geometric representation of the distal portion of the femur;
calculating a varus/valgus angle between a plane perpendicular to the mechanical axis of the femur and a plane passing through the distal-most point of the lateral portion and the AP line;
measuring an anterior-posterior distance between a posterior portion of a condyle of the femur and the intersection point;
positioning an anterior resection guide perpendicular to the AP line at a depth determined by the anterior-posterior distance between the posterior portion of the condyle of the femur and the intersection point; and
positioning a distal resection guide oriented according to the varus/valgus angle.

2. The method of claim 1, wherein the condyle of the femur is the medial condyle.

3. The method of claim 1, wherein the geometric representation is calculated from a point cloud.

4. The method of claim 1 wherein the geometric representation is calculated from an MRI.

5. The method of any of the above claims, wherein the depth of the distal resection is further adjusted according to flexion-extension balancing.

6. A system for resecting distal and anterior portions of a distal portion of a femur, comprising:
a geometric representation of the distal portion of the femur;
a virtual anterior resection plane within the geometric representation of the distal portion of the femur at a predetermined depth on the anterior portion of the distal femur, the virtual anterior resection plane being oriented at a predetermined angle relative to the femur in internal/external rotation wherein the virtual anterior resection plane includes an intersection point as a distal-most point of a lateral portion on the geometric representation of the virtual anterior resection plane;
an AP line on the geometric representation of the distal portion of the femur, the geometric representation having an anterior-posterior distance perpendicular to the AP line between a posterior portion of a condyle of the femur and the intersection point;
computer code configured to calculate a varus/valgus angle between a plane perpendicular to the mechanical axis of the femur and a plane passing through the intersection point and the AP line;
an anterior resection guide positioned perpendicular to the AP line at a depth determined by the anterior-posterior distance between the posterior portion of the condyle of the femur and the intersection point; and
a distal resection guide positioned according to the varus/valgus angle.

7. The system of claim 6, wherein the condyle of the femur is the medial condyle.

8. The system of claim 6, wherein the geometric representation is calculated from a point cloud.

9. The system of claim 6, wherein the geometric representation is calculated from an MRI.

10. The system of claim 6, wherein the depth of the distal resection is further adjusted according to flexion-extension balancing.

11. The system of claim 6, further comprising fiducials attached to the anterior and distal resection guides and fiducials attached to the femur.
